(19) United States
(12) Patent Application Publication
(10) Pub. No.: US 2026/0284362 A1
(43) Pub. Date: Sep. 24, 2026

(54) REMOVABLE ACCESS SHEATH

(71) Applicant: BOSTON SCIENTIFIC SCIMED, INC., Maple Grove, MN (US)

(72) Inventors: Jarrod Kenneth Neuharth, Coon Rapids, MN (US); Daniel Frank Massimini, Brooklyn Park, MN (US); Nicholas Lee Tassoni, Andover, MN (US)

(73) Assignee: BOSTON SCIENTIFIC SCIMED, INC., MAPLE GROVE, MN (US)

(21) Appl. No.: 19/566,077

(22) Filed: Mar. 13, 2026

Related U.S. Application Data
(60) Provisional application No. 63/775,504, filed on Mar. 21, 2025.

(51) Int. Cl. A61M 25/06; A61M 25/00; A61M 60/865

(52) U.S. Cl. CPC A61M 25/0668; A61M 25/0097; A61M 2025/0675; A61M 60/865

(57) ABSTRACT

Example access sheaths are disclosed. An example access sheath includes an elongate shaft having a distal end region, a proximal end region and a wall. The access sheath also includes a hub coupled to the proximal end region of the elongate shaft and a cutter releasably coupled to the hub, wherein the cutter is configured to translate along the elongate shaft from the proximal end region to the distal end region. Further, translation of the cutter along the elongate shaft cuts the wall of the elongate shaft from the proximal end region to the distal end region.

### CROSS-REFERENCE TO RELATED APPLICATION

This application claims the benefit of U.S. Patent Application Serial No. 63/775,504, filed Mar. 21, 2025, entitled "REMOVABLE ACCESS SHEATH”, which is incorporated by reference herein in its entirety.

### TECHNICAL FIELD

The disclosure relates generally to medical devices and more particularly to medical devices that are adapted for use in percutaneous medical procedures.

### BACKGROUND

In some instances, performing percutaneous medical procedures may require the insertion and/or maneuvering of relatively large medical devices through a patient’s vasculature. Therefore, in some instances a relatively large vascular access sheath may be utilized to facilitate the insertion of relatively large medical devices into a patient’s vasculature. However, inserting a relatively large medical device into the vasculature through a large bore access sheath for an extended time period may, for example, lead to tissue damage (e.g., leg ischemia) due to a reduction in blood flow. Tissue damage resulting from placement of a relatively large medical device into the vasculature for an extended time period may be lessened by minimizing the size of the access sheath used to access the vessel. It may be desirable to configure a relatively large bore access sheath capable of removal for replacement with a smaller access sheath.

### SUMMARY

This disclosure provides design, material, manufacturing method, and use alternatives for medical devices. An example access sheath includes an elongate shaft having a distal end region, a proximal end region and a wall. The access sheath also includes a hub coupled to the proximal end region of the elongate shaft and a cutter releasably coupled to the hub, wherein the cutter is configured to translate along the elongate shaft from the proximal end region to the distal end region. Further, translation of the cutter along the elongate shaft cuts the wall of the elongate shaft from the proximal end region to the distal end region.

Alternatively or additionally to any of the examples above, wherein the cutter includes a first grip member and a second grip member, wherein the first grip member includes a top surface and a bottom surface, and wherein the bottom surface faces a longitudinal axis of the elongate shaft.

Alternatively or additionally to any of the examples above, wherein the cutter further includes a blade assembly coupled to the bottom surface of the first grip member.

Alternatively or additionally to any of the examples above, wherein the blade assembly includes a blade having a cutting surface and guide member coupled to the blade.

Alternatively or additionally to any of the examples above, wherein the hub includes an inner aperture, and wherein the blade assembly is configured to be positioned within the aperture in a first position, and wherein the cutter is prevented from translating relative to the hub when the blade assembly is positioned within the aperture.

Alternatively or additionally to any of the examples above, wherein the first grip member is configured to shift from a first position to a second position, and wherein shifting the first grip member from the first position to the second position shifts the blade assembly out of the aperture and into an inner chamber of the hub.

Alternatively or additionally to any of the examples above, wherein shifting the blade assembly out of the aperture permits the cutter to translate relative to the hub, the elongate shaft or both the hub and the elongate shaft.

Alternatively or additionally to any of the examples above, wherein the guide member is configured to prevent the blade from being disengaged with the wall of the elongate shaft as the cutter translates along the elongate shaft.

Alternatively or additionally to any of the examples above, wherein the guide member includes a top surface configured to engage an inner surface of the elongate member as the cutter translates along the elongate shaft.

Alternatively or additionally to any of the examples above, wherein the cutter further includes a body having an aperture therein, and wherein the aperture is configured to permit the cutter to be removed from a medical device extending through at least a portion of the body.

Alternatively or additionally to any of the examples above 16, wherein the cutter includes a first groove and a second groove, and wherein the cutter is configured to be separated into a first cutter member and a second cutter member via tearing the cutter along the first groove and the second groove.

Alternatively or additionally to any of the examples above, wherein the hub is configured to separate into a first hub member and a second hub member.

Alternatively or additionally to any of the examples above, wherein the hub further includes a frangible region connecting a first hub member to a second hub member.

Alternatively or additionally to any of the examples above, wherein the frangible region may be configured to facilitate the separation of the hub into the first hub member and the second hub member, for example, configured to break to facilitate the separation of the hub into the first hub member and the second hub member.

Alternatively or additionally to any of the examples above, wherein the first hub member is coupled to the second hub member via a splitable membrane.

Alternatively or additionally to any of the examples above, further comprising a seal releasably coupled to the hub, wherein the seal is configured to be removed from a medical device extending through a portion of the seal.

Another access sheath for a medical device includes an elongate shaft having a distal end region, a proximal end region and a wall. The access sheath also includes a hub coupled to the proximal end region of the elongate shaft, wherein the hub includes a first hub member configured to separate from a second hub member. The access sheath also includes a cutter releasably coupled to the hub, wherein the cutter is configured to translate along the elongate shaft from the proximal end region to the distal end region. The hub also includes a seal releasably coupled to the hub, wherein the seal is configured to be removed from a medical device extending through a portion of the seal. Further, wherein translation of the cutter along the elongate shaft cuts the wall of the elongate shaft from the proximal end region to the distal end region.

Alternatively or additionally to any of the examples above, wherein the cutter includes a first grip member and a second grip member, wherein the first grip member includes a top surface and a bottom surface, and wherein the bottom surface faces a longitudinal axis of the elongate shaft.

Alternatively or additionally to any of the examples above, wherein the cutter further includes a blade assembly coupled to the bottom surface of the first grip member.

A method for advancing a medical device into a body lumen includes positioning an access sheath adjacent a body lumen, wherein the access sheath includes an elongate shaft having a distal end region, a proximal end region and a wall. The access sheath also includes a hub coupled to the proximal end region of the elongate shaft and a cutter releasably coupled to the hub, the cutter including a blade configured to cut the wall of the elongate shaft. The method also includes advancing the medical device through the access sheath and into the body lumen, translating the cutter along the elongate shaft from the proximal end region to the distal end region, wherein translation of the cutter along the elongate shaft cuts the wall of the elongate shaft from the proximal end region to the distal end region and removing the access sheath from the medical device via passing at least a portion of the medical device through the cut portion of the elongate shaft.

The above summary of some examples is not intended to describe each disclosed embodiment or every implementation of the present disclosure. The Figures and Detailed Description, which follow, more particularly exemplify these examples.

### BRIEF DESCRIPTION OF DRAWINGS

FIG. 1 is a perspective view of an example medical device;

FIG. 2A is a perspective view of an example access sheath;

FIG. 2B is a perspective view of an example access sheath;

FIG. 3 is a perspective view of the distal end region of an access sheath;

FIG. 4 is a cross-sectional view of an example access sheath in a first configuration;

FIG. 5 is a cross-sectional view of an example access sheath in a second configuration;

FIG. 6 is a perspective view of an example cutter;

FIG. 7 is a perspective view of a blade assembly;

FIG. 8A is a cross-sectional view illustrating actuation of a cutter to cut an elongate sheath of the access sheath of FIG. 2A;

FIG. 8B is a cross-sectional view illustrating actuation of a cutter to cut an elongate sheath of the access sheath of FIG. 2A;

FIG. 9A is a cross-sectional view illustrating actuation of a cutter to cut an elongate sheath of the access sheath of FIG. 2B;

FIG. 9B is a cross-sectional view illustrating actuation of a cutter to cut an elongate sheath of the access sheath of FIG. 2B;

FIG. 9C is a cross-sectional view illustrating actuation of a cutter to cut an elongate sheath of the access sheath of FIG. 2B;

FIG. 9D is a cross-sectional view illustrating actuation of a cutter to cut an elongate sheath of the access sheath of FIG. 2B;

FIG. 10 is a cross-sectional view illustrating actuation of a cutter to cut an elongate sheath of the access sheath;

FIG. 11 is a perspective view illustrating actuation of a cutter to cut an elongate sheath of an access sheath;

FIG. 12 is a partial exploded view of the example access sheath;

FIG. 13 is a perspective view of another example access sheath;

FIG. 14 is a portion of the access sheath of FIG. 13 in a first configuration;

FIG. 15 is a portion of the access sheath of FIG. 13 in a second configuration;

FIG. 16 is a perspective view of a portion of another example access sheath in a first configuration;

FIG. 17 is a portion of the access sheath of FIG. 16 in a second configuration;

FIG. 18 is a perspective view of a portion of another example access sheath in a first configuration;

FIG. 19 is a perspective view of a portion of the example access sheath of FIG. 18;

FIG. 20 is a portion of the access sheath of FIG. 18 in a second configuration;

FIG. 21 illustrates a removable access sheath seal;

FIG. 22 illustrates a removable access sheath seal;

FIG. 23 illustrates a removable access sheath seal in a first configuration;

FIG. 24 illustrates the removable seal of FIG. 23 in a second configuration;

FIG. 25 illustrates a removable seal in a first configuration;

FIG. 26 illustrates the removable seal of FIG. 25 in a second configuration;

FIG. 27 illustrates a removable cutter in a first configuration; and

FIG. 28 illustrates the removable cutter of FIG. 27 in a second configuration.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some examples”, “other examples”, etc., indicate that the embodiment described may include one or more particular features, structures, and/or characteristics. However, such recitations do not necessarily mean that all examples include the particular features, structures, and/or characteristics. Additionally, when particular features, structures, and/or characteristics are described in connection with one embodiment, it should be understood that such features, structures, and/or characteristics may also be used connection with other examples whether or not explicitly described unless clearly stated to the contrary.

The following detailed description should be read with reference to the drawings in which similar elements in different drawings are numbered the same. The drawings, which are not necessarily to scale, depict illustrative examples and are not intended to limit the scope of the disclosure.

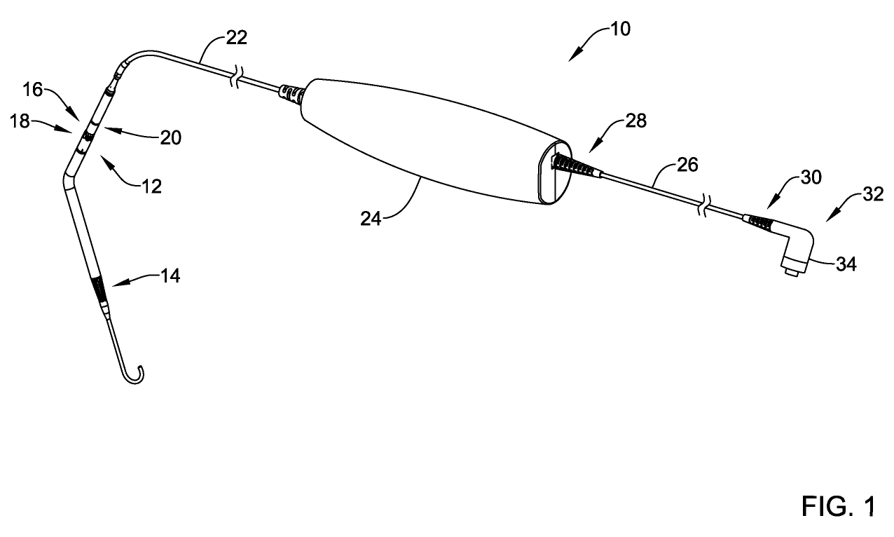

FIG. 1 shows an example percutaneous blood pump 10 (i.e., percutaneous cardiac assist device and/or circulatory support device) for connection to a medical device console (not shown). In the cases of blood pumps such as that depicted in FIG. 1, percutaneous cardiac assist devices and/or circulatory support devices support the pumping action of the heart. These devices may be positioned percutaneously into a patient’s heart, such as disposed across a valve opening such as, for example, an aortic valve. Such percutaneous blood pumps 10 assist the heart in pumping blood throughout the cardiovascular system, and the present disclosure provides uses, devices, and methods incorporating such devices as will be described herein. Such percutaneous blood pumps 10 may be connected to a medical device console for operating the blood pump and/or monitoring the operation of the blood pump and/or one or more patient parameters. However, a wide variety of medical devices are utilized to treat patients which are connected to a medical device console for operating the medical device and/or monitoring the operation of the medical device and/or one or more patient parameters. Accordingly, aspects of the present disclosure may be incorporated into a wide variety of medical devices.

Typically, a circulatory support device such as the example blood pump 10 shown in FIG. 1 includes a blood pump housing 12 including a blood inlet 14 at a blood inflow end of the blood pump housing 12 and a blood outlet 16 at a blood outflow end of the blood pump housing 12. The blood pump housing 12 may include an impeller 18 or other blood pumping device configured to convey blood through the blood pump housing 12 from the blood inlet 14 to the blood outlet 16. In some instances, the impeller 18 may be disposed within an impeller housing portion of the blood pump housing 12. The impeller 18 may be constructed of radially oriented blades protruding from a drive shaft and/or drive mechanism, for example. In some instances, the blood inlet 14 may be positioned and/or be positionable in a ventricle of the patient, while the blood outlet 16 may be positioned and/or be positionable in the aorta of the patient, such that the blood pump 10 pumps blood across the aortic valve. The impeller 18 may be driven by a motor 20 or other means of rotating the impeller 18 during operation thereof. In some instances, the motor 20 may be housed within a motor housing portion of the blood pump housing 12. In other instances, the motor 20 may be arranged at another location, if desired. For example, the motor 20 may be located on the blood pump 10 such that it remains outside the body when the blood pump 10 is in use. The motor 20 or other means of rotating the impeller 18 may be configured to rotate the impeller 18 through magnetism, electromagnetism, mechanical force, electrical force, and/or any combination or permutation of the aforementioned, such that the drive shaft and/or drive mechanism rotates the blades of the impeller 18, thereby creating a blood flow to propel blood through the blood pump housing 12 and beyond into the cardiovascular system. The blood pump 10 may include an elongate catheter shaft 22 extending proximally from the blood pump housing 12 for navigating the blood pump 10 to a desired location within the cardiovascular system of the patient. The catheter shaft 22 may extend proximally to a junction box 24, a handle, a manifold, or other structure of the blood pump 10 configured to remain exterior of the patient.

Furthermore, the blood pump 10 may include a cable 26 at a proximal end thereof having a connection interface 32 (e.g., connector) at an end thereof. The cable 26 may have a first, distal end 28 connected to the junction box 24, a handle, a manifold, or other structure of the blood pump 10, and a second, proximal end 30 having the connection interface 32 including a connector 34 that is configured to be connected to a medical device console (e.g., blood pump console). The cable 26 may include one or more of, an electrical pathway for powering the motor 20, an electrical pathway for powering one or more sensors, an electrical pathway for transmitting signals from one or more sensors associated with the blood pump 10, a fiber optic filament for transmitting signals from one or more sensors associated with the blood pump 10, or other communication conduits. In some instances, the cable 26 and associated connector 34, may be considered a single-connector cable device providing the only connection between the blood pump 10 and a medical device console controlling the blood pump 10.

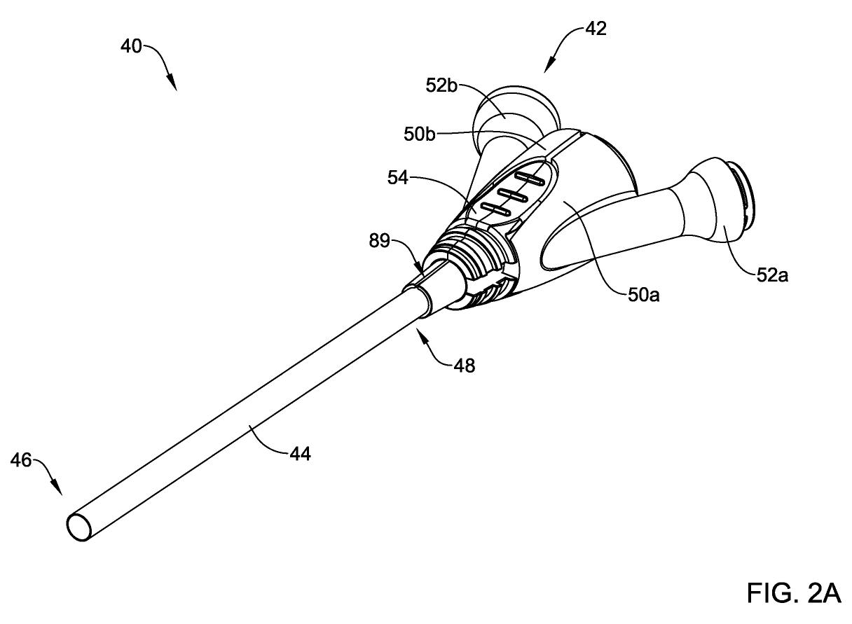

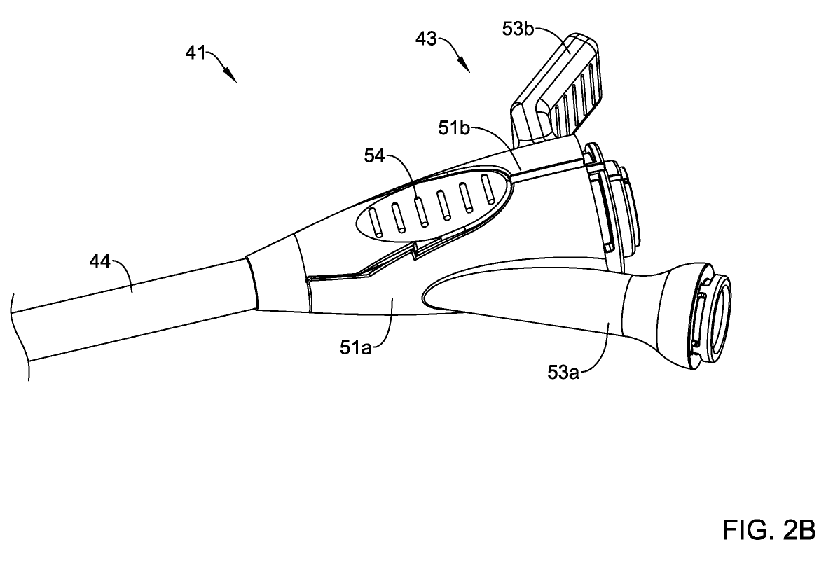

FIG. 2A is a perspective view of an example removable access sheath 40 for use in providing vascular access for introducing a medical device (e.g., the percutaneous blood pump of FIG. 1) into a vasculature at a vascular access site. The access sheath 40 may include a splitable hub 42. The access sheath 40 may also include an elongate shaft 44 having a distal end region 46 and a proximal end region 48 coupled to the hub 42. The access sheath 40 may also include a removeable cutter 54 coupled to the hub 42.

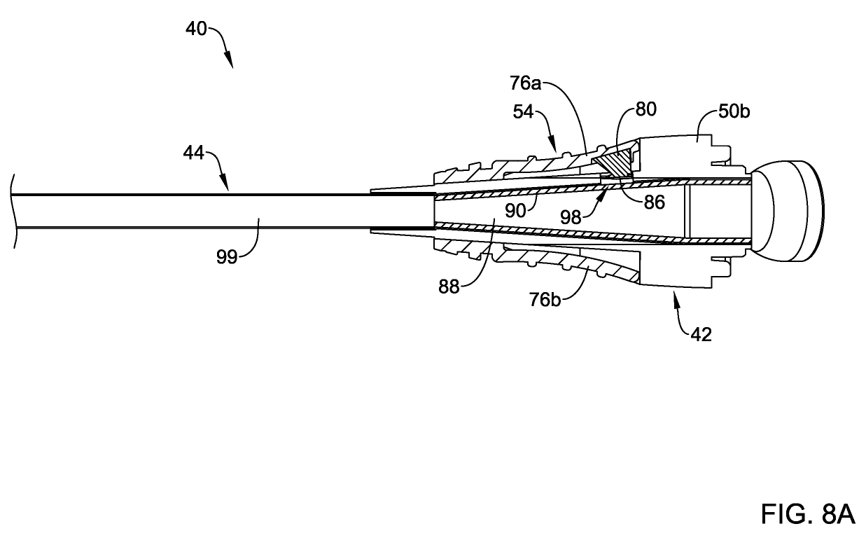

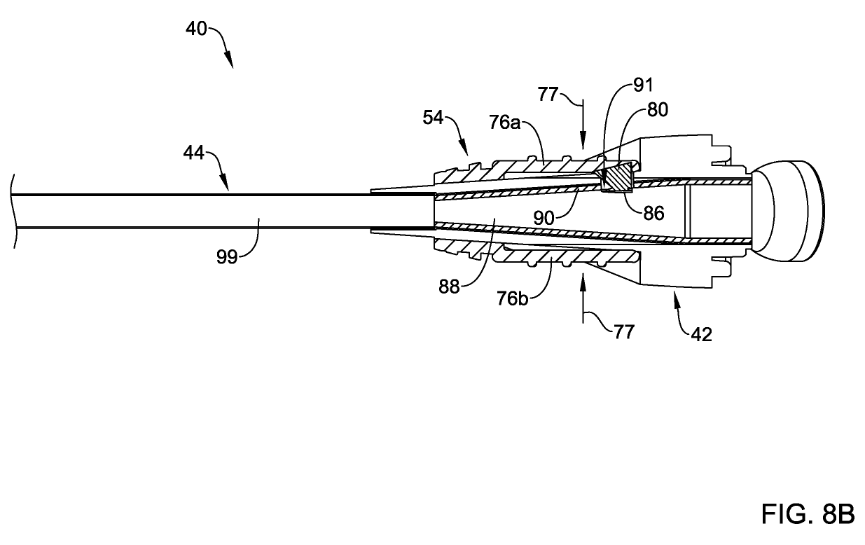

The splitable hub 42 may include a first hub member 50a and a second hub member 50b releasably attached together. In some examples, the first hub member 50a and the second hub member 50b may be releasably attached to one another via a splitable membrane 90 (shown in FIG. 8A). In some examples, the splitable membrane 90 attaching the first hub member 50a and the second hub member 50b may be formed from a soft durometer material. The hub 42 may also include a first side port 52a and a second side port 52b. In some examples, the hub 42 may only include the first side port 52a. In some examples, the first side port 52a and a second side port 52b may be configured to permit a user to grasp each of the first side port 52a and a second side port 52b to separate the first side port 52a and the second side port 52b. In some instances, the first side port 52a may extend at an acute angle from the first hub member 50a and the second side portion 52b may extend at an acute angle from the second hub member 50b. The first side port 52a and the second side port 52b may provide access to one or more lumens extending through the elongate shaft 44.

FIG. 2B is a perspective view of an example removable access sheath 41 for use in providing vascular access for introducing a medical device (e.g., the percutaneous blood pump of FIG. 1) into a vasculature at a vascular access site. The access sheath 41 may be similar in form and function to the access sheath 40 described herein. The access sheath 41 may include an elongate shaft 44 (similar in form and function to the elongate shaft 44) coupled to a hub 43. The access sheath 41 may also include a removeable cutter 54 (similar in form and function to the cutter 54) coupled to the hub 43.

The splitable hub 43 may include a first hub member 51a and a second hub member 51b releasably attached together. The first hub member 51a and the second hub member 51b may be releasably attached to one another. In some examples, the hub 43 may also include a side port 53a and a grip member 53b. In some examples, the hub 43 may only include the side port 53a. In some examples, the side port 53a and a grip member 53b may be configured to permit a user to grasp each of the side port 53a and the grip member 53b to separate the side port 53a and the grip member 53b. In some instances, the side port 53a may extend at an acute angle from the first hub member 51a and the grip member 53b may extend at an acute angle from the second hub member 51b. The side port 53a and the grip member 53b may provide access to one or more lumens extending through the elongate shaft 44.

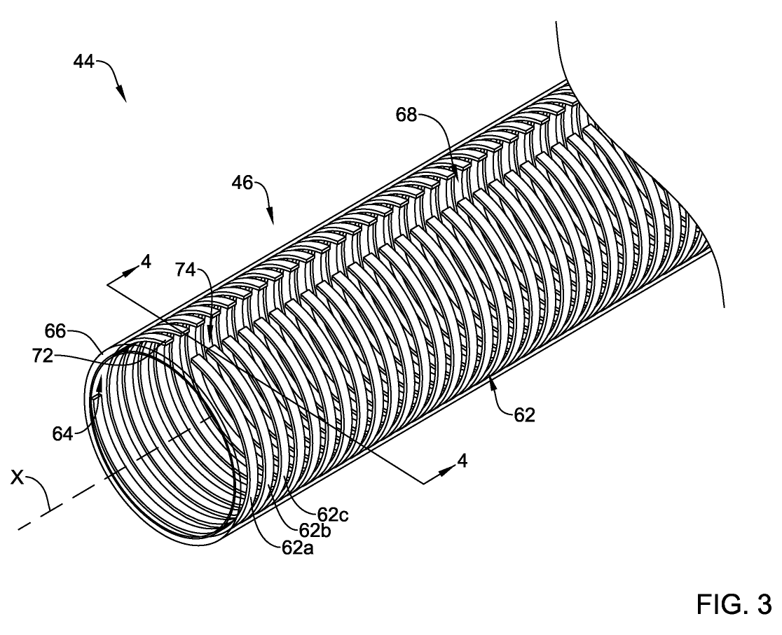

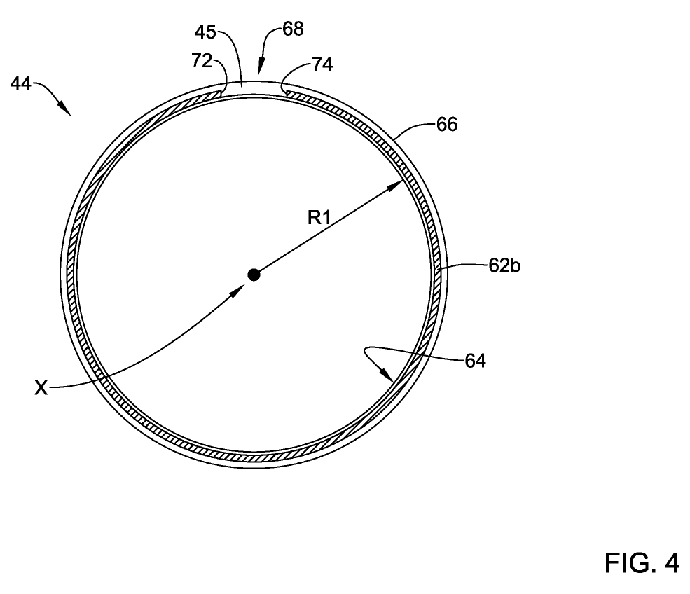

FIG. 3 illustrates the distal end region 46 of the example removeable access sheath 40 (e.g., delivery sheath, access sheath, etc.). FIG. 3 illustrates that the elongate shaft 44 of the access sheath 40 may include a plurality of reinforcing members 62 aligned along the elongate shaft 44 from the proximal end region 51 to the distal end region 46 of the elongate shaft 44. Further, it can be appreciated from FIG. 3 that each respective reinforcing member 62 may be positioned adjacent to one another along a longitudinal axis X of the elongate shaft 44. For example, FIG. 3 illustrates three individual reinforcing members 62a, 62b, 62c positioned adjacent to one another and aligned along the longitudinal axis X of the elongate shaft 44. FIG. 3 further illustrates that each of the plurality of reinforcing members 62 may partially extend about the longitudinal axis X of the elongate shaft 44. For example, FIG. 3 illustrates that each of the respective reinforcing members 62 may extend only partially around the longitudinal axis X of the elongate shaft, whereby each of the plurality of reinforcing members includes a first end circumferentially spaced away from a second end. For example, FIG. 3 illustrates that the reinforcing member 62b includes a first end 72 circumferentially spaced away from a second end 74. In some examples, the portion of the elongate shaft 44 positioned between the first ends (e.g., first end 72) and the second ends (e.g., second end 74) of each reinforcing member 62 may define a cutting region 68 along the elongate shaft. As will be described in greater detail below, the cutting region 68 may include the region along the elongate shaft 44 in which the cutter 54 may cut through a wall (shown in FIG. 4 and which may include the outer layer 66 and the inner layer 64) of the elongate shaft 44.

As will be described in greater detail below, FIG. 3 illustrates that the elongate shaft 44 may further include a lubricious liner 64 extending from the distal end region 46 to the proximal end region 51. It can be appreciated from FIG. 3 that the liner 64 may be positioned radially inward of the plurality of the reinforcing members 62. It can be appreciated that, in some examples, an outer surface of the lubricious liner 64 may contact and be attached to an inner surface of the plurality of reinforcing members 62.

Additionally, the access sheath 40 may also include an outer layer 66 Outer layer 66 may extend along (e.g., cover) both the plurality of reinforcing members 62 and the liner 64. As will be discussed in greater detail below, the plurality of reinforcing members 62 may be positioned between (e.g., sandwiched between) the lubricious liner 64 and the outer layer 66. In some examples, the outer layer 66 may be attached to both the liner 64 and the plurality of reinforcing members 62. In some examples, the proximal region of the liner 64 and/or the proximal end region of the outer layer 66 may be attached to the hub 42.

FIG. 4 illustrates an example cross-sectional view of the elongate shaft 44 taken along the line 4-4 of FIG. 3. As discussed herein, FIG. 4 illustrates the reinforcing member 62b positioned between (e.g., sandwiched between) the inner lubricious liner 64 and the outer layer 66. FIG. 4 further illustrates that the inner lubricious liner 64 may extend continuously around the longitudinal axis X of the elongate shaft 44. FIG. 4 further illustrates that the elongate shaft 44 may include a radius R₁

Additionally, FIG. 4 illustrates that the reinforcing member 62b extends partially around the longitudinal axis X of the elongate shaft 44. For example, FIG. 4 illustrates that the elongate member 62b is configured such that a first end 72 of the reinforcing member 62b is spaced away from the second end 74 of the reinforcing member 62b. In some examples, the first end and the second end of each respective reinforcing member 62 may be longitudinally offset. For example, the first end 72 of the reinforcing member 62b may be longitudinally offset from the second end 74 of the reinforcing member 62b. As discussed herein, FIG. 4 also illustrates the cutting region 68 positioned between the first end 72 and the second end 74 of the reinforcing member 62b.

As discussed herein, the elongate sheath 44 may be configured to permit the cutter to cut through a wall 45 of the elongate sheath along the cutting region 68. For example, the access sheath 40 described herein may be utilized to insert and position a relatively large medical device (e.g., blood pump 10) within a patient. However, after inserting and positioning a large medical device (e.g., blood pump 10), it may be desirable to remove the access sheath 40 from the medical device (e.g., remove it from the shaft 22 of the blood pump 10 which may be extending through the access sheath 40). Accordingly, a user may utilize the cutter 54 (shown in FIG. 2) to cut through the wall 45 of the elongate shaft 44 of the access sheath 40, thereby purposefully splitting the wall 45 of the elongate shaft 44 along the cutting region 68 from the proximal end region 51 to the distal end region 46 of the elongate shaft 44.

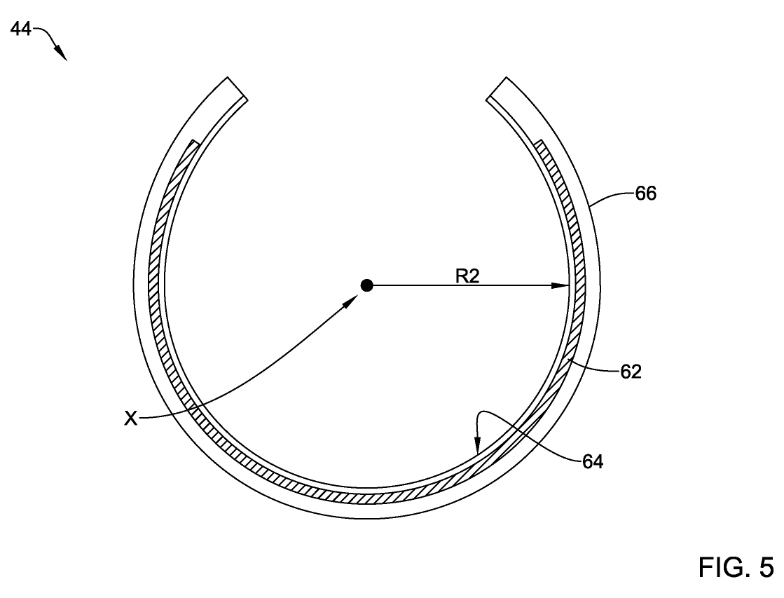

Further, in some examples, each of the reinforcing members 62 of the access sheath 40 may be configured to be positioned between the liner 64 and the outer layer 66 (such as that shown in FIG. 4) under circumferential tension. In other words, during the manufacturing process, the reinforcing members 62 as shown in FIG. 4 may be placed under circumferential tension while the liner 64 and the outer layer 66 are formed around each respective reinforcing member 62, thereby maintaining each reinforcing members 62 under circumferential tension until the cutter 54 is utilized to cut the wall of the elongate shaft 44. It can be appreciated that the term circumferential tension, as used herein, may describe a first configuration in which the reinforcement member 62 is held under tension in a first position (as shown in FIG. 4) and whereby each reinforcement member 62 may be biased to expand radially outward to a second position (as shown in FIG. 5) when the elongate shaft 44 is cut, thereby releasing each reinforcement member 62 from the first configuration and allowing each reinforcement member 62 to expand radially outward to the second position shown in FIG. 5. FIG. 5 further illustrates that the elongate shaft 44 may include a radius R₂ in the second configuration which is greater than the radius R₁ of the elongate shaft 44 shown in the first configuration of FIG. 4. In some examples, each reinforcement member 62 may be described as having an outward radial self-bias defined by the configuration illustrated in FIG. 5.

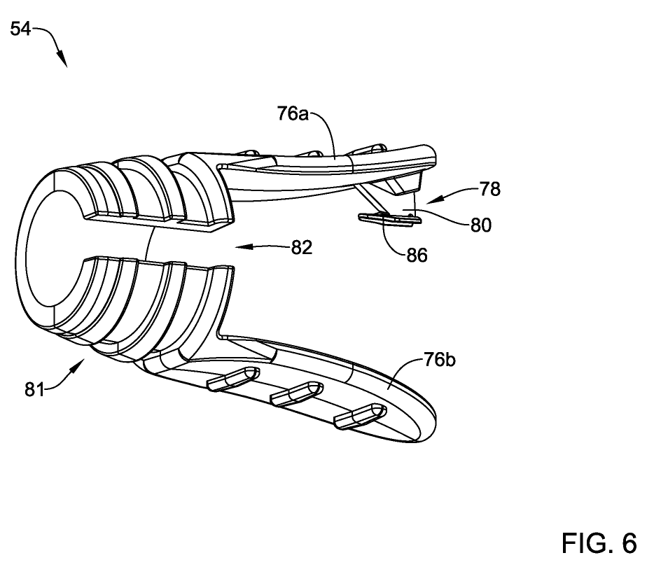

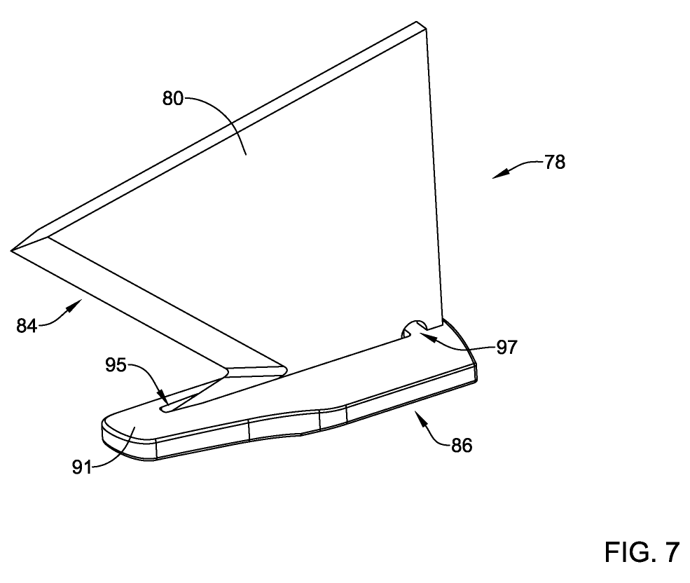

FIG. 6 illustrates a cutter 54 of the access sheath 40 and FIG. 7 is a perspective view of a blade assembly 78 described herein. FIG. 6 illustrates that the cutter 54 may include a body portion 81. Further, the body portion 81 may include a longitudinally-extending opening 82 extending through a wall of the body portion 81. Accordingly, the body portion 81 may be generally “C-shaped” when the cutter 54 is viewed from the distal end. As will be discussed in greater detail below, the opening 82 may be utilized to remove the cutter from another medical device (e.g., guidewire, etc.) used in conjunction with the access sheath 40 during a medical procedure.

FIG. 6 further illustrates that the cutter 54 may further include a first grip member 76aand a second grip member 76b, whereby each grip member 76a, 76b may extend away from the body portion 81 in a proximal direction. It can be appreciated that the cutter 54 may be configured such that a user may grasp the grip members 76a, 76b with their fingers to manipulate the cutter 54. It can be appreciated that, in some examples, each grip member 76a, 76b may include one or more ergonomic features to aid a user in grasping and manipulating the cutter 54.

FIG. 6 further illustrates that the cutter 54 may further include a cutting assembly 78 attached to an inner facing surface (e.g., underside) of the grip member 76a. FIGS. 6-7 illustrate that the cutting assembly 78 may include a cutting blade 80 having a distally facing cutting surface 84. FIG. 6 illustrates that the cutting blade 80 may extend vertically downward from the underside of the first grip member 76a, thereby positioning the cutting surface 84 such that it faces toward the elongate shaft 44. While FIG. 6 illustrates the cutting assembly 78 attached to the first grip member 76a, it can be appreciated that, in some examples, the cutting assembly 78 may be attached to the second grip member 76b. In yet other examples, the cutter 54 may be configured such that a first cutting assembly 78 is attached to the first grip member 76a and a second cutting assembly is attached to the second grip member 76b. It can be appreciated that the first grip member 76a may include a top surface facing away from the longitudinal axis X of the access sheath 40 and a bottom surface facing toward the longitudinal axis X of the access sheath 40. Additionally, the second grip member 76b may include a top surface facing away from the longitudinal axis X of the access sheath 40 and a bottom surface facing toward the longitudinal axis X of the access sheath 40.

FIGS. 6-7 further illustrate that the cutting assembly 78 may further include a guide member 86 coupled to the blade 80. As illustrated in FIG. 7, the guide member 86 may include an aperture 95 configured to permit a portion of the blade 80 to extend therein. The guide member 86 may also include a pivot region 97 which may permit the guide member 86 to pivot, flex and/or rotate relative to the blade 80. Further, it can be appreciated that the aperture 95 further aids the guide member 86 in pivoting, flexing and/or rotating relative to the blade 80, as the aperture 95 permits a portion of the blade 80 to extend past a top surface 91 of the guide member 86 and into the body of the guide member 86.

FIG. 8A illustrates a cross-sectional view of a portion of the access sheath 40. FIG. 8A illustrates the removable cutter 54 removably coupled to the second hub member 50b of the hub 42. It can be appreciated from FIG. 8A that the hub 42 may include an inner lumen 88 which is in contact with an inner lumen 99 of the elongate shaft 44. FIG. 8A further illustrates the first grip member 76a and the second grip member 76b in a pre-cutting configuration, whereby neither the first grip member 76a and the second grip member 76b have been actuated to initiate translation of the cutter 54 along either the hub 42 or elongate shaft 44. In some examples, the first hub member 51aand the second hub member 51b may be releasably attached to one another via a splitable membrane 90. In some examples, the splitable membrane 90 attaching the first hub member 50a and the second hub member 50b may be positioned along an inner surface of the first hub member 50a and the second hub member 50b of the hub 42. In some examples, the splitable membrane 90 attaching the first hub member 50a and the second hub member 50b may be formed from a soft durometer material.

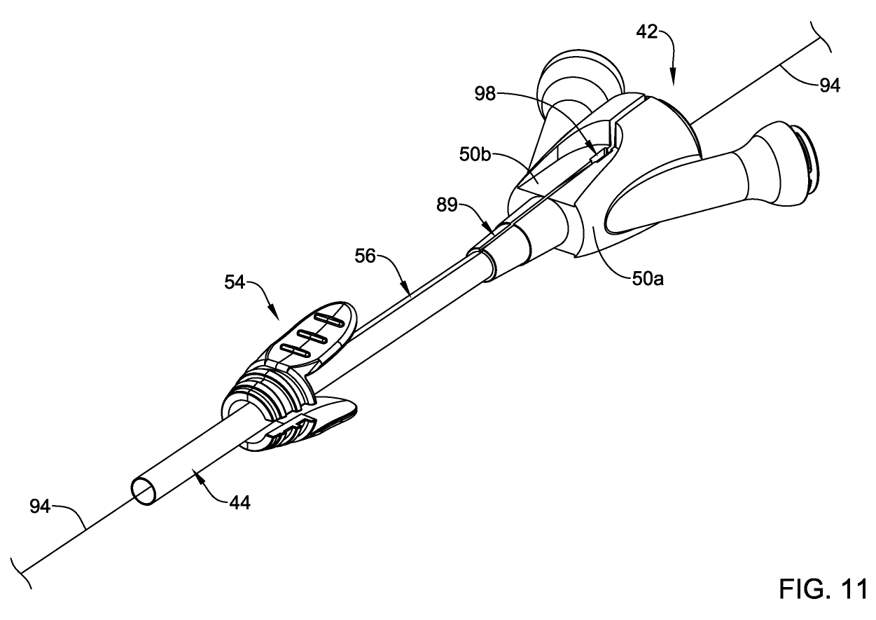

FIG. 8A further illustrates that the guide member 86 may be aligned with a vertical aperture 98 (shown in FIG. 11) positioned within the hub 42 (within an inner chamber of the hub 42). The vertical aperture 98 may be formed by a combination of recesses in both the first hub member 50a (shown in FIG. 2) and the second hub member 50b. It can be appreciated that the surfaces of the first hub member 50a (shown in FIG. 2) and the second hub member 50b which form the vertical aperture 98 may prevent the cutter 54 from translating in a proximal-to-distal direction as the distal region of the guide member 86 may contact the surfaces of the first hub member 50a (shown in FIG. 2) and the second hub member 50b which form the vertical aperture 98.

FIG. 8B illustrates the actuation (e.g., pinching, squeezing, etc.) of the first grip member 76a and the second grip member 76b relative to the hub 42. The actuation of the first grip member 76a and the second grip member 76b is illustrated by the directional arrows 77 in FIG. 8B. As discussed herein, it can be appreciated that a user may use both of their hands to actuate the cutter 54 relative to the hub 42. For example, a user may grasp and pinch the first grip member 76a and the second grip member 76b of the cutter 54 with the fingers of one hand while holding onto the hub 42 with the fingers of their other hand. FIG. 8B further illustrates that the pinching of the first grip member 76a and the second grip member 76b may translate the cutting assembly 78 downward within the aperture of the 98 (shown in FIG. 8A), whereby the downward translation of the cutting assembly 78 (including the first grip member 76a) punctures the membrane splitable membrane 90. In other words, the downward translation of the cutting assembly 78 drives the first grip member 76a through the splitable membrane 90, thereby shifting the guide member 86 from a position within the aperture 98 to a position within the lumen 88 within the hub 42, thereby freeing the guide member 86 from within the aperture 98.

Additionally, FIG. 8B illustrates that the downward translation of the cutting assembly 78 (via the pinching of the first grip member 76a and the second grip member 76b) may shift the guide member 86 from a position within the aperture 98 to a position within the lumen 88 within the hub 42, thereby freeing the guide member 86 from within the aperture 98. Because the blade 80 may be sized such that its width may permit it to fit within a longitudinally-extending channel 89 (shown in FIG. 2) of the hub 42, once the guide member 86 is translated downward and freed from the aperture 98 (via puncturing the splitable membrane 90), the cutter 54 may be free to translate in a proximal-to-distal direction within the hub 42. It can be appreciated that once the cutter 54 begins the proximal-to-distal translation within the hub 42, a top surface 91 of the guide member 86 may contact an inner surface splitable membrane 90, thereby preventing the first grip member 76a and the second grip member 76b from expanding radially outward and pulling the blade 80 out of the channel 89 (shown in FIG. 2).

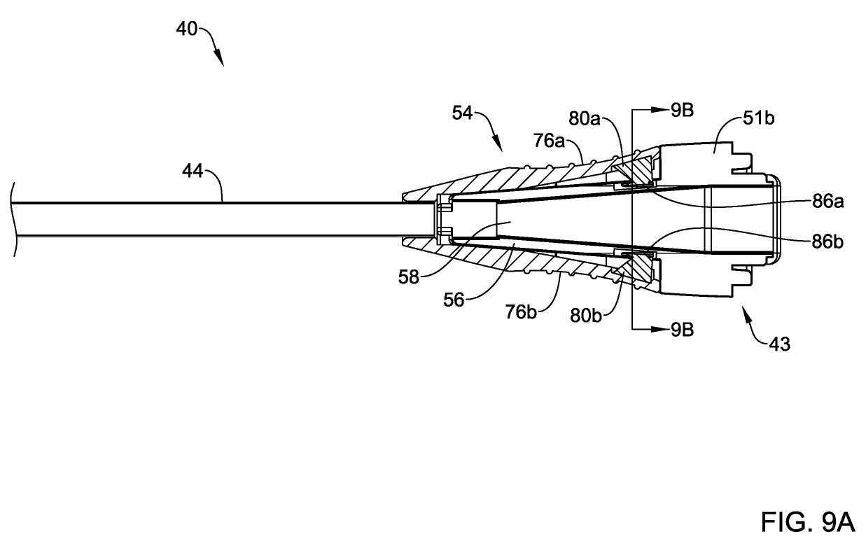

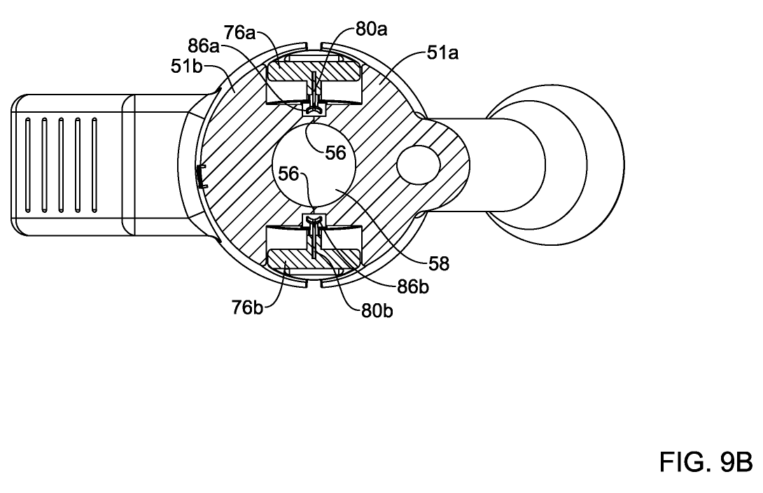

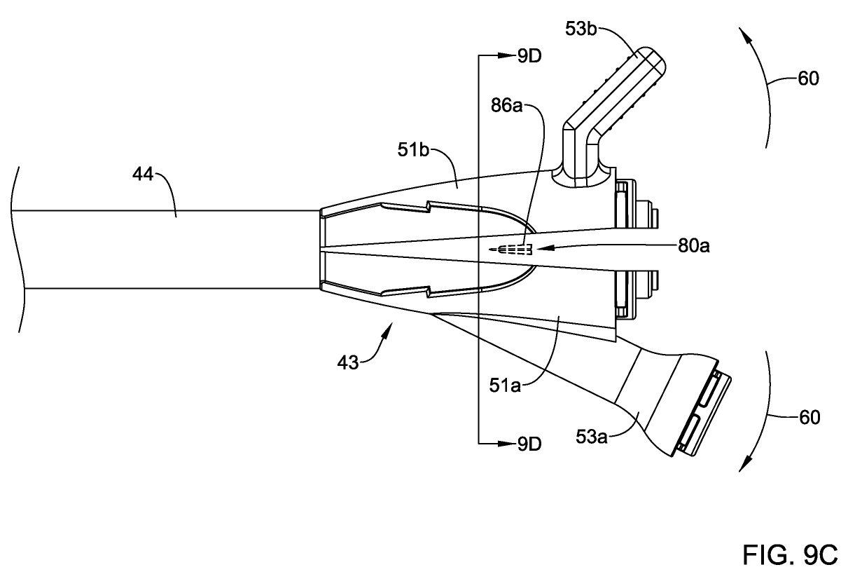

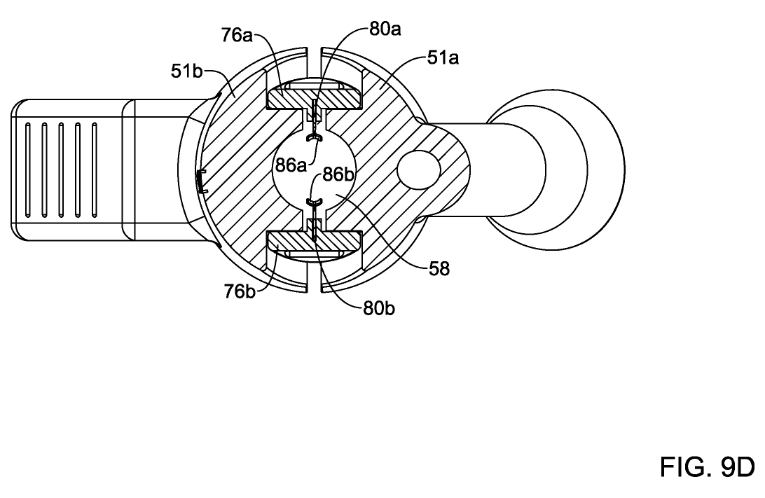

FIG. 9A illustrates a cross-sectional view of a portion of the access sheath 41 shown in FIG. 2B. FIG. 9A illustrates the removable cutter 54 removably coupled to the first hub member 51a (hidden from view in the cross-sectional view of FIG. 9A) and the second hub member 51b of the hub 43 (also shown in FIG. 3). FIG. 8A further illustrates the first grip member 76a and the second grip member 76bin a pre-cutting configuration, whereby neither the first grip member 76a nor the second grip member 76b have been actuated to initiate translation of the cutter 54 along either the hub 43 or elongate shaft 44. In some examples, the first hub member 51aand the second hub member 51b may be releasably attached to one another along a frangible region 56. In some examples, the frangible region 56 attaching the first hub 51a and the second hub member 51b may include a portion of the hub 43 which extends from the first hub member 51a to the second hub member 51b, thereby connecting the first hub member 51a to the second hub member 51b. In other words, the first hub member 51a, the second hub member 51b and the frangible region 56 may be formed of a homogeneous structure that defines the hub 43. As will be described in greater detail below, the hub 43 may be designed to permit a user to break (e.g., crack) splitable portion 56 to separate the first hub member 51a and the second hub member 51b. FIG. 9A also illustrate an inner lumen 58 of the hub 43.

FIG. 9A further illustrates that the cutter 54 may include a first blade 80acoupled to the first grip member 76a and a first guide member 86a and a second blade 80b coupled to the second grip member 76b and a second guide member 86b. The first blade 80aand the second blade 80bmay be similar in form and function to the blade 80 described herein. The first guide member 86aand the second guide member 86b may be similar in form and function to the guide member 86 described herein. FIG. 9A illustrates that the first blade 80a may be circumferentially offset from the second blade 80b.

FIG. 9B illustrates a cross-sectional view taken along line 9B-9B of FIG. 9A. FIG. 9B illustrates the removable cutter 54 removably coupled to the first hub member 51a and the second hub member 51b of the hub 43 (also shown in FIG. 3). FIG. 9B further illustrates the first grip member 76a and the second grip member 76bin a pre-cutting configuration, whereby neither the first grip member 76a nor the second grip member 76b have been actuated to initiate the translation of the first blade 80a and the second blade 80b toward the inner lumen 58. FIG. 9B illustrates the first hub member 51a and the second hub member 51b attached along the frangible region 56.

FIG. 9C illustrates a top view of the hub 43 attached to the elongate shaft 44. FIG. 9C illustrates the breaking of the hub 43 along the frangible region 56. It can be appreciated that the hub 43 may be configured to permit a user to grasp and pull the side port 53a and the grip member 53b away from one another to break the hub 43 along the frangible region, thereby separating the first hub member 51a and the second hub member 51b from one another. The breaking and separation of the first hub member 51a from the second hub member 51b is depicted by the directional arrows 60 in FIG. 9C. FIG. 9C shows the position of the first blade 80a and the guide member 86a relative to the first hub member 51a and the second hub member 51b after the first hub member 51a and the second hub member 51b have been separated (it is noted that the first grip member 76a has been omitted from FIG. 9C for clarity purposes).

FIG. 9D illustrates a cross-sectional view taken along line 9D-9D of FIG. 9C. FIG. 9D illustrates the translation of the first blade 80a and the second blade 80b toward the inner lumen 58. It can be appreciated that the pinching of the first grip member 76a and the second grip member 76b may translate the cutting assembly 78 downward toward the lumen 58, whereby the translation of the blade 80a (and guide member 86a) positions the blade 80a adjacent to the wall 45 (shown in FIG. 4) of the sheath 44 and the translation of the blade 80b (and guide member 86b) positions the blade 80b adjacent to the wall 45 (shown in FIG. 4) of the sheath 44. It can be appreciated that the configuration shown in FIG. 9D may permit the cutter 54 (including the blades 80a, 80b and the guide members 86a, 86b) in a proximal-to-distal translation from the hub 43 toward the elongate sheath 44.

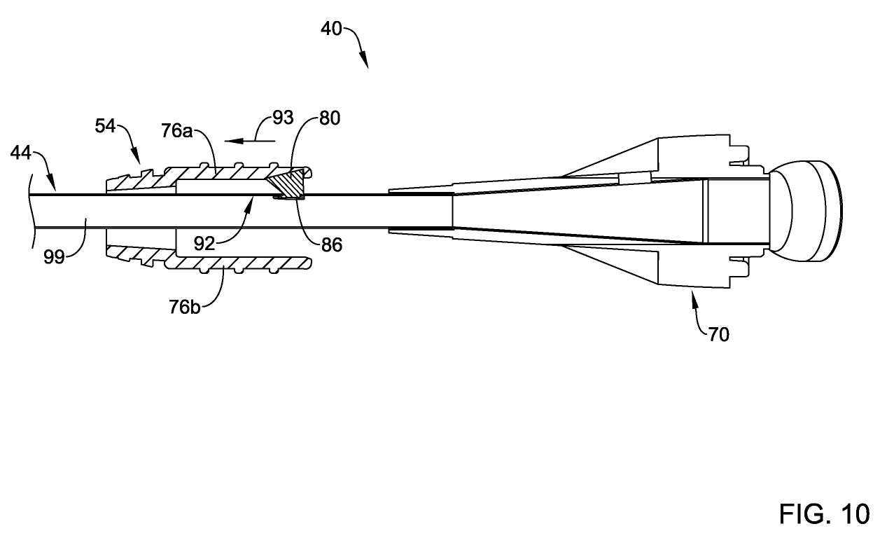

FIG. 10 illustrates the cutter 54 being translated in a proximal-to-distal direction to initiate cutting of the elongate tube 44 along the cutting region 68 (shown in FIGS. 3-4). Translation of the cutter 54 in a proximal-to-distal direction is depicted by the directional arrow 93 in FIG. 10. FIG. 10 illustrates that translation of the cutter 54 in a proximal-to-distal direction may permit the cutting surface 84 of the blade 80 to engage and cut the wall of the inner elongate member 44. Additionally, it can be appreciated from FIG. 10 that the access sheath 40 may be configured such the inner surface 90 (FIG. 9) within a hub 70 (whereby the hub 70 may represent any hub disclosed herein, including the hub 42 and the hub 43 disclosed herein) is circumferentially aligned with an inner surface 92 of the elongate shaft 44. Accordingly, as the cutter 54 is translated in a proximal-to-distal direction, the top surface 91 (shown in FIG. 7) of the guide member 86 (or guide member 86a shown in FIGS. 9A-9D) may contact the inner surface 92 of the elongate shaft 44, thereby preventing the first grip member 76a and the second grip member 76b from expanding radially outward and pulling the blade 80 (or the blades 80a, 80b shown in FIGS. 9A-9D) out of the wall of the elongate shaft 44 and thereby allowing the cutting surface 84 of the blade 80 (or the blades 80a, 80b shown in FIGS. 9A-9D) to cut the elongate shaft 44.

FIG. 11 is a perspective view of the cutter 54 being translated in a proximal-to-distal direction to initiate cutting of the elongate shaft 44 along the cutting region 68 (shown in FIGS. 3-4). FIG. 11 illustrates that translation of the cutter 54 in a proximal-to-distal direction may permit the cutting surface 84 of the blade 80 to engage and cut the wall of the inner elongate member 44 (the cut portion of the elongate tube 44 is depicted by the reference numeral 56 in FIG. 11). As described herein, FIG. 11 further illustrates that the cut portion 56 of the elongate shaft 44 is aligned with the channel 89 formed between the first hub member 50a and the second hub member 50b. FIG. 11 also illustrates a guidewire 94 extending through the elongate shaft 44 and the hub 42.

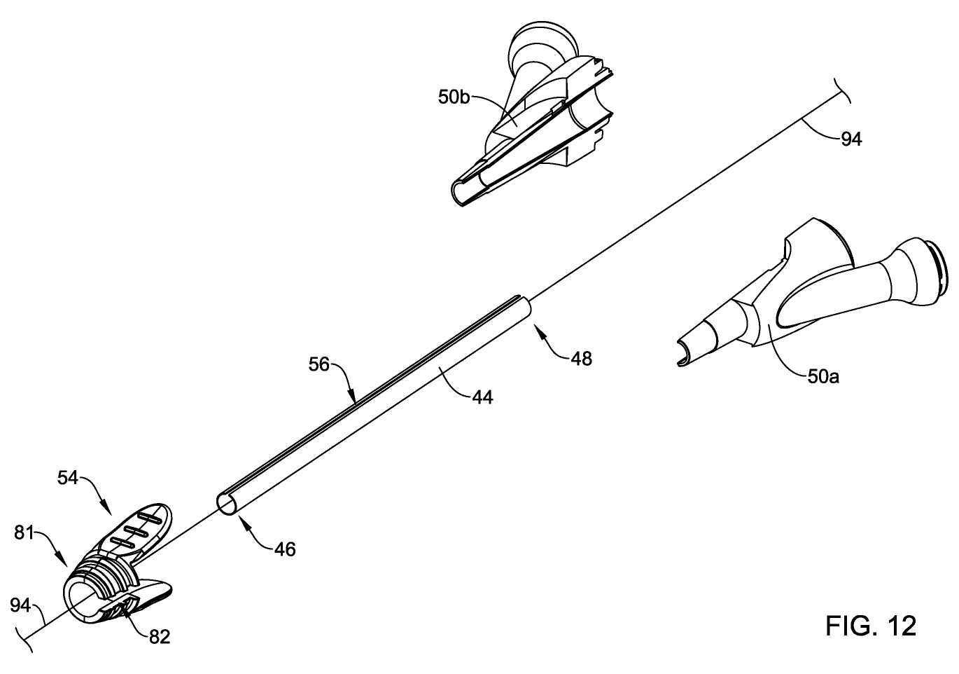

FIG. 12 illustrates the cutter 54 having been translated in a proximal-to-distal direction along the entire length of the elongate shaft 44. Accordingly, FIG. 12 illustrates that the cutter 54 has engaged and cut the wall of the inner elongate shaft 44 from a proximal end region 48 of the elongate shaft 44 to the distal end region 46 of the elongate shaft 44. The cut portion of the elongate shaft 44 is depicted by the reference numeral 56 in FIG. 12. It can be appreciated that the cut portion 56 may permit the elongate shaft 44 to be removed from the guidewire 94 (or other portion of a medical device) shown in FIG. 12.

FIG. 12 further illustrates that the first hub member 50a may be separated from the second hub member 50b. As discussed herein, the first hub member 50a and the second hub member 50b may be releasably attached to one another via a splitable membrane. In some examples, the splitable membrane (such as the membrane 90 shown in FIG. 8A) attaching the first hub member 50a and the second hub member 50b may be formed from a soft durometer material which may be configured to split when the first hub member 50a is pulled away from the second hub member 50b. It can be further appreciated that separating the first hub member 50a from the second hub member 50b may permit the hub 42 to be removed from the guidewire 94 (or other portion of a medical device) shown in FIG. 12.

Additionally, FIG. 12 illustrates the longitudinally-extending opening 82 extending through a wall of the body portion 81 of the cutter 54. It can be appreciated that the longitudinally-extending opening 82 may permit the elongate shaft 44 to be removed from the guidewire 94 (or other portion of a medical device) show in FIG. 12.

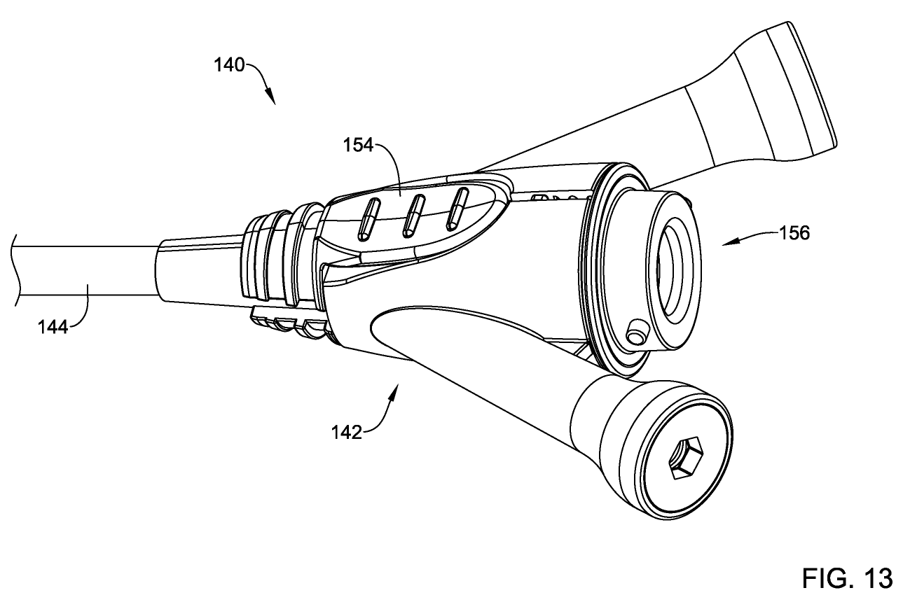

FIG. 13 illustrates another example access sheath 140. The access sheath 140 may be similar in form and function to the access sheath 40. FIG. 13 illustrates that the access sheath 140 may include a seal member 156. In some examples, the seal member 156 may include a hemostasis valve. The seal member 156 may be configured to seal and minimize fluid loss during interventional and diagnostic procedures. For example, the seal member 156 may be configured to seal off and minimize fluid loss during interventional and diagnostic procedures when a guidewire (e.g., guidewire 94 shown in FIGS. 11-12) is extending through the seal member 156.

It can be appreciated that any of the access sheaths described herein may be configured to include one or more safety mechanisms which are configured to prevent a cutter 154 from inadvertently being removed from a hub 142, whereby the cutter 154 could inadvertently initiate cutting of an elongate shaft 144. It can be appreciated that it may be desirable to configure the access sheaths described herein to include one or more safety mechanisms which require a user to purposely manipulate (e.g., actuate, disengage, activate) a safety mechanism as a prerequisite step to remove the cutter 154 from the hub 142 and initiate cutting of the elongate tube 144.

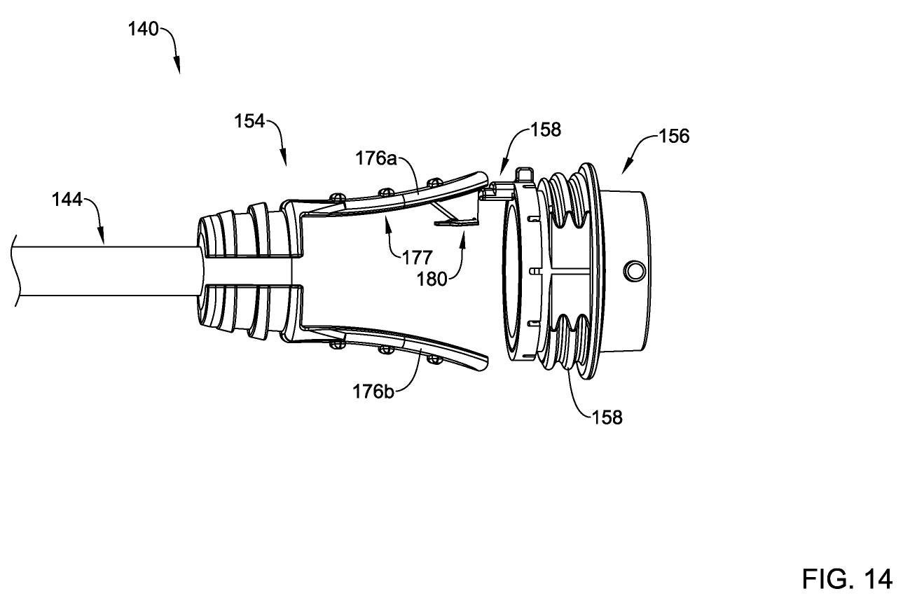

FIG. 14 illustrates the access sheath 140 of FIG. 13 whereby the hub member 142 has been omitted for clarity purposes. FIG. 14 illustrates that the seal member 156 may include a threaded region 158 for mechanical engagement with a mating threaded region on the hub 142. FIG. 14 further illustrates that the seal member 156 may further include a projection 158 which may be configured to be positioned adjacent the bottom surface 177 (e.g., underside) of the first grip member 176a. It can be appreciated that FIG. 14 may illustrate the relative position of the seal member 156 and the cutter 154 (which is positioned on the hub 142 as shown in FIG. 13) when the seal member 156 is fully set (e.g., screwed) into the hub 142. Accordingly, it can be appreciated that the projection 158 prevents the actuation (e.g., pinching) of the first grip member 176aand the second grip member 176b, thereby preventing the cutter 154 from inadvertently being removed from the hub 142.

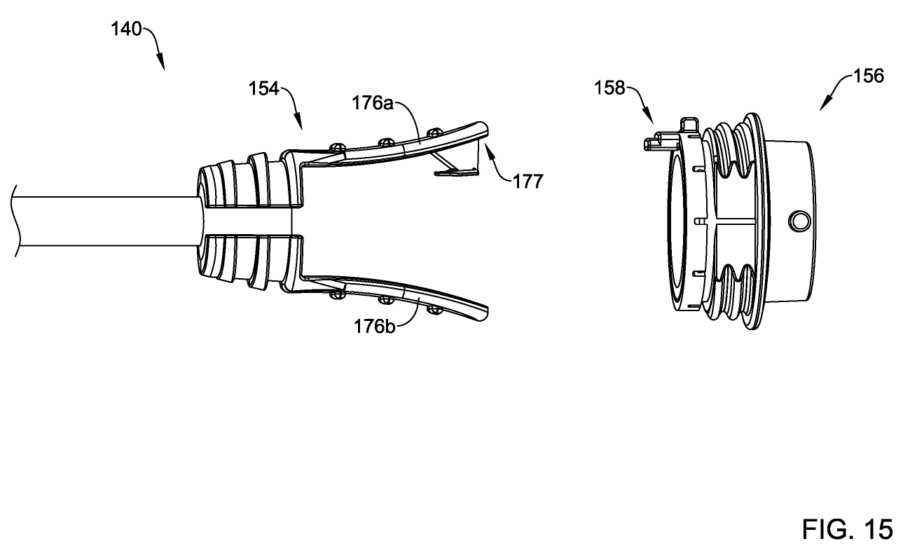

FIG. 15 illustrates the access sheath of FIGS. 13-14 whereby the seal member 156 has been unscrewed and spaced away from the cutter 154 (which is positioned on the hub 142 as shown in FIG. 13). It can be appreciated that unscrewing and removing the seal member 156 from the hub 142 may permit the actuation (e.g., pinching) of the first grip member 176a and the second grip member 176b (because the projection is no longer preventing the movement of the first grip member 176a), thereby permitting the cutter 154 to be translated in a proximal-to-distal direction to engage and cut the elongate tube 144. It can be appreciated that the configuration of the seal member 156 illustrated in FIGS. 13-15 may act as a safety mechanism which requires a user to purposely remove the seal member 156 from the hub 142 as a perquisite step to remove the cutter 154 from the hub 142 and initiate cutting of the elongate tube 144.

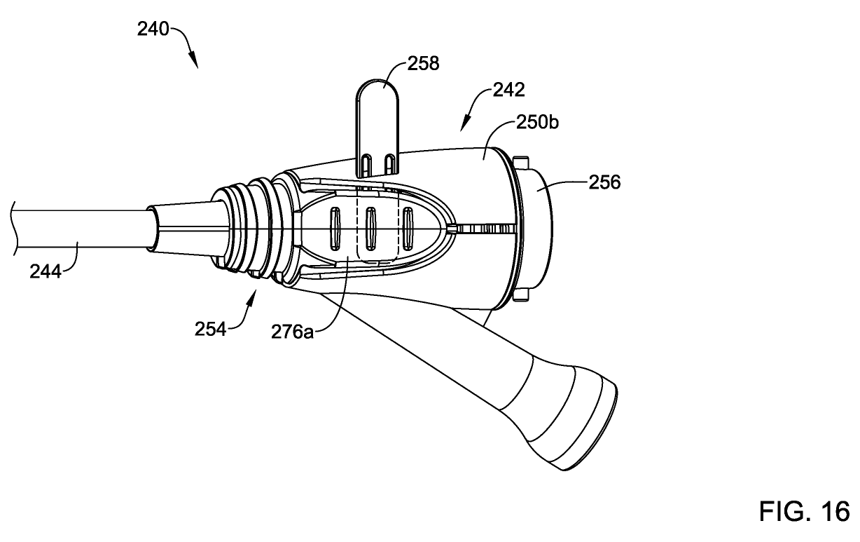

FIG. 16 illustrates another example access sheath 240. The access sheath 240 may be similar in form and function to the access sheath 40. FIG. 16 illustrates that the access sheath 240 may include a seal member 256. In some examples, the seal member 256 may include a hemostasis valve. The seal member 256 may be configured to seal off and minimize fluid loss during interventional and diagnostic procedures. For example, the seal member 256 may be configured to seal off and minimize fluid loss during interventional and diagnostic procedures when a guidewire (e.g., guidewire 94 shown in FIGS. 11-12) is extending through the seal member 256. FIG. 16 further illustrates that the access sheath 240 may further include a safety tab 258 extending through the wall of a second hub member 250b. FIG. 16 further illustrates that the safety tab 258 may extend through the wall of the second hub member 250b such that it is positioned underneath (e.g., along the underside) of a first grip member 276aof a cutter 254. Accordingly, it can be appreciated that the safety tab 258 may be configured to prevent the actuation (e.g., pinching) of the first grip member 276a, thereby preventing the cutter 254 from inadvertently being removed from a hub 242.

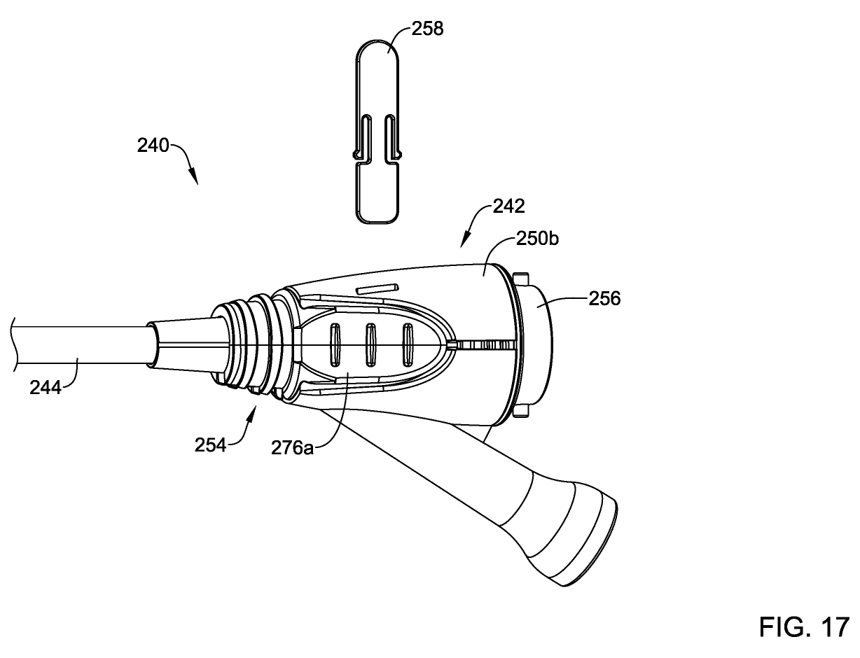

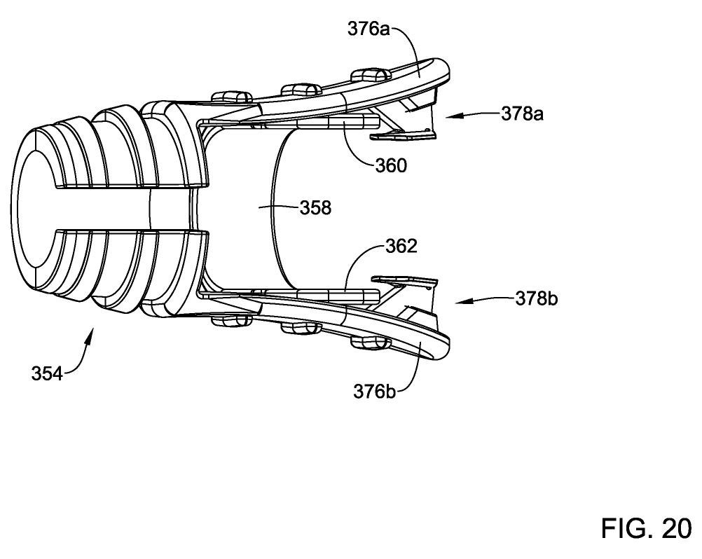

FIG. 17 illustrates the access sheath of FIG. 16 whereby the safety tab 258 has been removed and spaced away from the second hub member 250b. It can be appreciated that removing the safety tab 258 from the second hub member 250b may permit the actuation (e.g., pinching) of the first grip member 276a(because the safety tab 258 is no longer preventing the downward movement of the first grip member 276a), thereby permitting the cutter 254 to be translated in a proximal-to-distal direction to engage and cut an elongate shaft 244. It can be appreciated that the configuration of the safety tab 258 illustrated in FIGS. 16-17 may act as a safety mechanism which requires a user to purposely remove the (shown in FIG. 20) safety tab 258 from the second hub member 250b as a perquisite step to remove the cutter 254 from the hub 242 and initiate cutting of the elongate shaft 244.

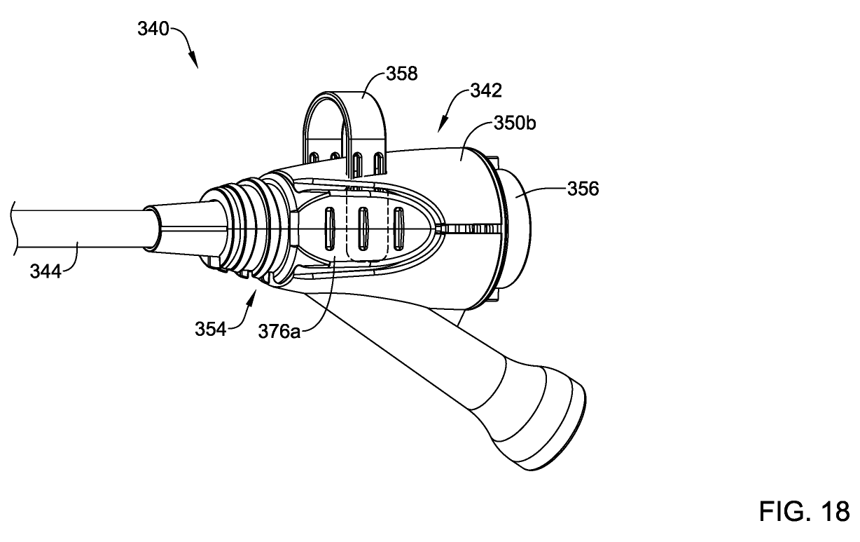

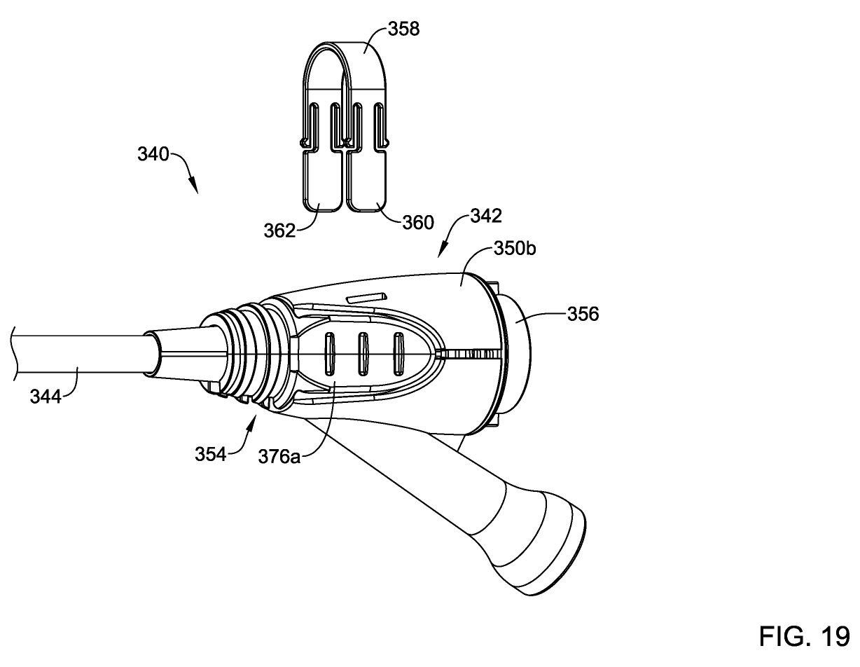

FIG. 18 illustrates another example access sheath 340. The access sheath 340 may be similar in form and function to the access sheath 40. FIG. 18 illustrates that the access sheath 340 may include a seal member 356. In some examples, the seal member 356 may include a hemostasis valve. The seal member 356 may be configured to seal off and minimize fluid loss during interventional and diagnostic procedures. For example, the seal member 356 may be configured to seal off and minimize fluid loss during interventional and diagnostic procedures when a guidewire (e.g., guidewire 94 shown in FIGS. 11-12) is extending through the seal member 356. FIG. 18 further illustrates that the access sheath 340 may further include a safety clip 358 extending through the wall of the second hub member 350b. FIG. 18 further illustrates that the safety clip 358 may be generally U-shaped and include a first end region 360 and a second end region 362 (FIG. 19), each of which extend through the wall of the second hub member 350b such that the first end region 360 is positioned underneath (e.g., along the underside) of the first grip member 376aand the second end region 362 is positioned underneath (e.g., along the underside) of the second grip member 376b(shown in FIG. 20) of a cutter 354. The positioning of the first end region 360 underneath (e.g., along the underside) the first grip member 376aand the second end region 362 underneath (e.g., along the underside) the second grip member 376b(shown in FIG. 20) of the cutter 354 is shown in FIG. 19. Accordingly, it can be appreciated that the safety clip 358 may be configured to prevent the actuation (e.g., pinching) of the first grip member 376aand the second grip member 376b (shown in FIG. 20), thereby preventing the cutter 354 from inadvertently being removed from a hub 342.

FIG. 19 illustrates the access sheath of FIG. 18 whereby the safety clip 358 has been removed and spaced away from the second hub member 350b. It can be appreciated that removing the safety clip 358 from the second hub member 350b may permit the actuation (e.g., pinching) of the first grip member 376a and the second grip member 376b (because the safety clip 358 is no longer preventing the movement of the first grip member 376a and the second grip member 376b), thereby permitting the cutter 354 to be translated in a proximal-to-distal direction to engage and cut an elongate shaft 344. It can be appreciated that the configuration of the clip 358 illustrated in FIGS. 18-19 may act as a safety mechanism which requires a user to purposely remove the safety clip 358 from the second hub member 350b as a perquisite step to remove the cutter 354 from the hub 342 and initiate cutting of the elongate shaft 344.

FIG. 20 illustrates an example of a cutter 354 which includes two cutting assemblies 378a, 378b. For example, FIG. 20 illustrates a first cutting assembly 378a attached to the first grip member 376a and a second cutting assembly 378b attached to the second grip member 376b. It can be appreciated that actuation of the cutter 354 may permit cutting the elongate shaft 344 along two cutting regions which may be circumferentially offset from one another approximately 180 degrees.

It can be appreciated that it may be desirable to configure any of the seal members described herein to be removable from a guidewire or other medical device extending through the seal member.

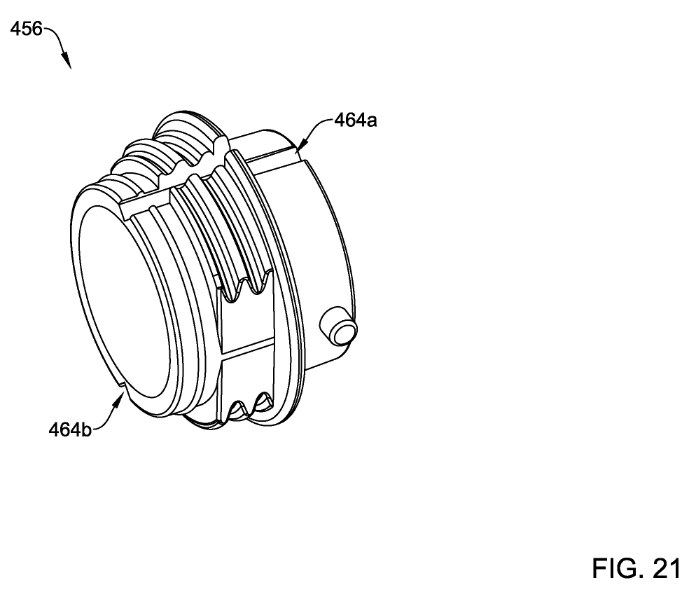

FIG. 21 illustrates an example removeable seal 456. FIG. 21 illustrates that the seal 456 may include a first groove 464aand a second groove 464b. FIG. 21 illustrates that the first groove 464a may be circumferentially offset from the second groove 464b approximately 180 degrees. Further, it can be appreciated that the first groove 464a, the second groove 464b or both the first groove 464a and the second groove 464b may be configured to spit, tear, rip, etc., thereby permitting the seal 456 to be removed from a guidewire or other medical device extending through the seal 456.

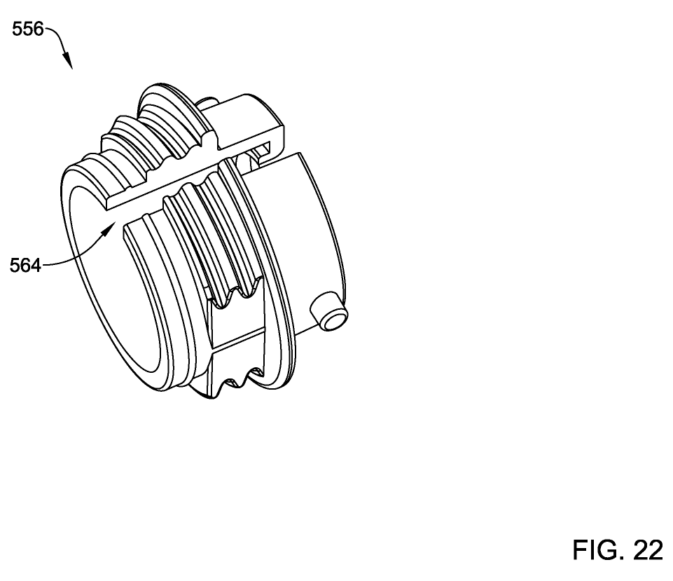

FIG. 22 illustrates another removeable seal 556. FIG. 22 illustrates that the seal 556 may include a channel 564. It can be appreciated that the channel 564 may be configured to permit the seal 556 to be removed from a guidewire or other medical device extending through the seal 556.

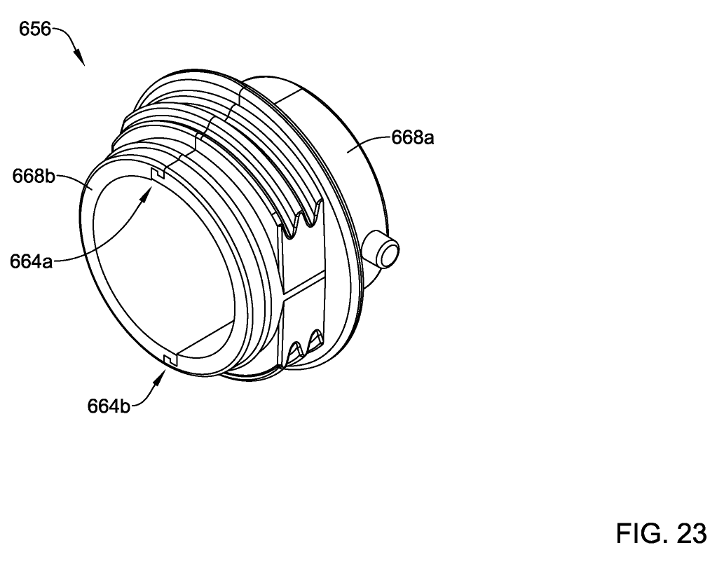

FIG. 23 illustrates another removeable seal 656. FIG. 23 illustrates that the seal 656 may include a first seal member 668areleasably coupled to a second seal member 668b. FIG. 23 illustrates that the first seal member 668a may be releasably coupled to the second seal member 668b via a first engagement region 664a and a second engagement region 664b.

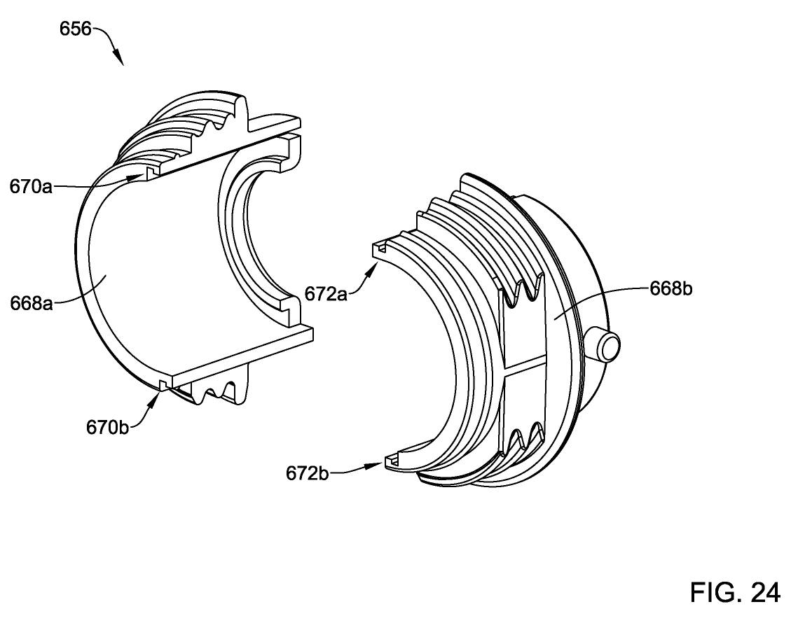

FIG. 24 illustrates the first seal member 668a separated and spaced away from the second seal member 668b. FIG. 24 illustrates the first engagement region 664a may include a longitudinally extending projection 670a(e.g., lip) on the first seal member 668awhich may be configured to engage a longitudinally extending recess 672a positioned on the second seal member 668b. Similarly, FIG. 24 illustrates the second engagement region 664b may include a longitudinally extending projection 670b(e.g., lip) on the first seal member 668awhich may be configured to engage a longitudinally extending recess 672b positioned on the second seal member 668b.

It can be appreciated that separating the first seal member 668a from the second seal member 668b may permit the seal member 656 to be removed from a guidewire or other medical device extending through the seal member 656.

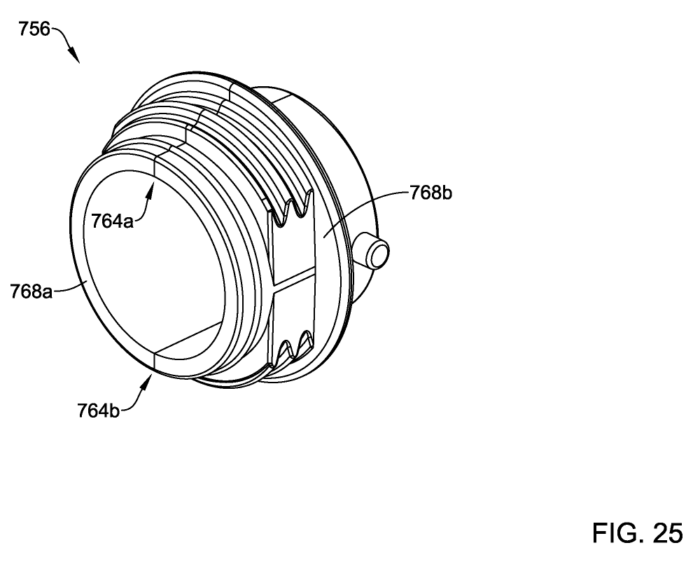

FIG. 25 illustrates another removeable seal 756. FIG. 25 illustrates that the seal 756 may include a first seal member 768areleasably coupled to a second seal member 768b. FIG. 25 illustrates that the first seal member 768a may be releasably coupled to the second seal member 768b via a first engagement region 764a and a second engagement region 764b.

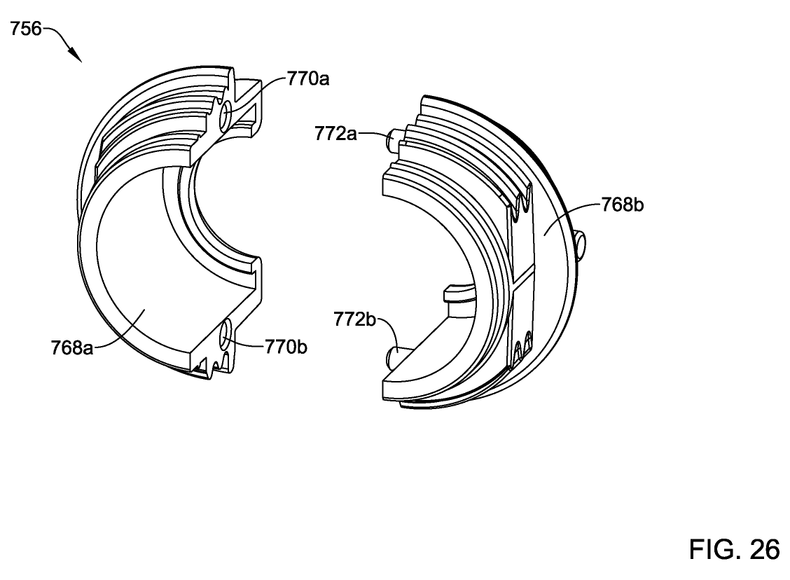

FIG. 26 illustrates the first seal member 768a separated and spaced away from the second seal member 768b. FIG. 24 illustrates first seal member 768a may be coupled and aligned with the second seal member 768b via engagement of a first pin 772a positioned on the second seal member 768b within a first recess 770a positioned on the first seal member 768a and a second pin 772b positioned on the second seal member 768b within a second recess 770b positioned on the first seal member 768a.

It can be appreciated that separating the first seal member 768a from the second seal member 768b may permit the seal member 756 to be removed from a guidewire or other medical device extending through the seal member 756.

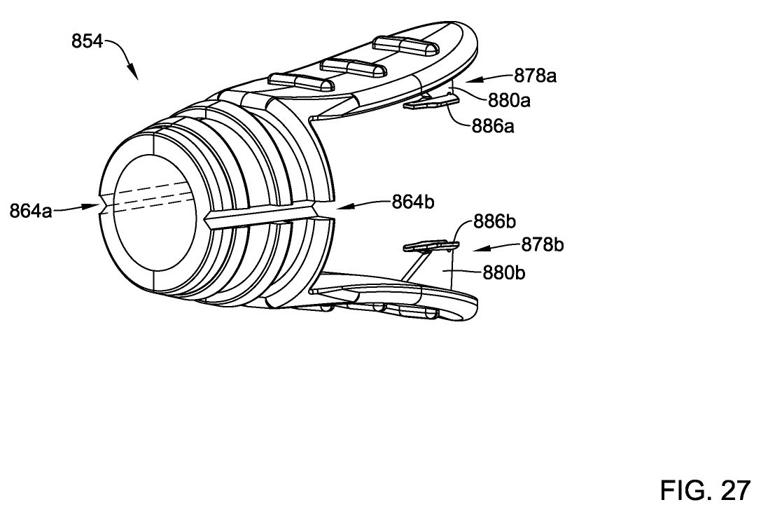

FIG. 27 illustrates an example removable cutter 854 which may be utilized in any of the access sheath configurations disclosed herein. The cutter 854 may be similar in form and function to the cutter 54. For example, the cutter 854 may include a first cutting assembly 878a attached to and/or extending from an inner facing surface (e.g., underside) of a first grip member and/or a second cutting assembly 878b attached to and/or extending from an inner facing surface (e.g., underside) of a second grip member. The first cutting assembly 878a may include a cutting blade 880a having a distally facing cutting surface. The first cutting blade 880a may extend vertically downward from the underside of the first grip member, thereby positioning the cutting surface of the cutting blade 880a such that it faces toward the elongate shaft 44. In some instances, the first cutting assembly 878a may include a guide member 886a coupled to the cutting blade 880a. The guide member 886a, similar to other guide members described herein, may extend laterally from the lateral sides of the cutting blade 880a.

When provided therewith, the second cutting assembly 878b may include a cutting blade 880b having a distally facing cutting surface. The second cutting blade 880b may extend vertically upward from the underside of the second grip member, thereby positioning the cutting surface of the cutting blade 880b such that it faces toward the elongate shaft 44. In some instances, the second cutting assembly 878bmay include a guide member 886bcoupled to the cutting blade 880b. The guide member 886b, similar to other guide members described herein, may extend laterally from the lateral sides of the cutting blade 880b.

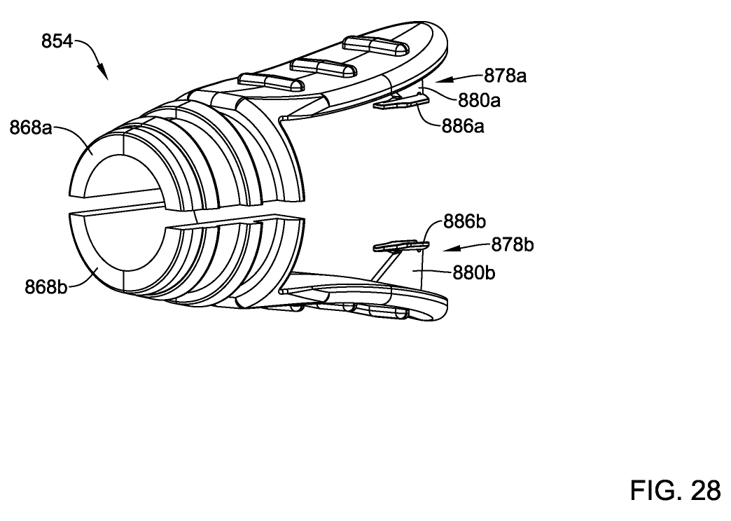

FIG. 27 illustrates that the cutter 854 may include a first groove 864aand a second groove 864b. FIG. 27 illustrates that the first groove 864a may be circumferentially offset from the second groove 864b approximately 180 degrees. The first and second grooves 864a, 864b may be configured to facilitate separating the cutter 854 into two separate pieces. For instance, as illustrated in FIG. 28, it can be appreciated that the first groove 864a, the second groove 864b or both the first groove 864a and the second groove 864b may be configured to break, spit, tear, rip, etc., thereby permitting a first cutter member 868a, which may include the first grip member and/or the first cutting blade 880a, to separate from a second cutter member 868b, which may include the second grip member and/or the second cutting blade 880b, and allowing the cutter 854 to be removed from a guidewire or other medical device extending through the cutter 854.

In some examples, access sheath 40 (or other access sheaths and components thereof described herein) may be made from materials such as metals, metal alloys, polymers, ceramics, metal-polymer composites, or other suitable materials, and the like. Some examples of suitable materials may include metallic materials such as stainless steels (e.g. 304v stainless steel or 316L stainless steel), nickel-titanium alloys (e.g., nitinol, such as super elastic or linear elastic nitinol), nickel-chromium alloys, nickel-chromium-iron alloys, cobalt alloys, nickel, titanium, platinum, or alternatively, a polymeric material, such as a high performance polymer, or other suitable materials, and the like. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

In some examples, the access sheath 40 (or other access sheaths and components thereof described herein) may be made from materials such as, for example, a polymeric material, a ceramic, a metal, a metal alloy, a metal-polymer composite, or the like. Examples of suitable polymers may include nylons, acrylonitrile butadiene styrene (ABS), polyurethane, a polyether-ester such as ARNITEL® available from DSM Engineering Plastics, a polyester such as HYTREL® available from DuPont, a linear low density polyethylene such as REXELL®, a polyamide such as DURETHAN® available from Bayer or CRISTAMID® available from Elf Atochem, an elastomeric polyamide, a block polyamide/ether, a polyether block amide such as PEBA available under the trade name PEBAX®, silicones, polyethylene, Marlex high-density polyethylene, polyetheretherketone (PEEK), polyimide (PI), and polyetherimide (PEI), a liquid crystal polymer (LCP) alone or blended with other materials. In some examples, a suitable polymeric material may have a yield strain of at least 20%, at least 30%, at least 40%, at least 50%, or more. In some examples, the sheath, the membrane, the liner or other components of the access sheaths disclosed herein may be made from a material having a low coefficient of friction. In some examples, the sheath, the membrane, the liner or other components of the access sheaths disclosed herein may be formed from a fluoropolymer, such as polytetrafluoroethylene (PTFE) or fluorinated ethylene propylene (FEP).

Portions of access sheath 40 (or other access sheaths and components thereof described herein) may be made of, may be doped with, may include a layer of, or otherwise may include a radiopaque material. Radiopaque materials are understood to be materials capable of producing a relatively bright image on a fluoroscopy screen or another imaging technique such as X-ray during a medical procedure. This relatively bright image aids the user of device in determining its location. For example, one or more of the elements described above (i.e., the sheath, the membrane, the medical device, etc.) may include or be formed from a radiopaque material. Suitable materials can include, but are not limited to, bismuth subcarbonate, iodine, gold, platinum, palladium, tantalum, tungsten or tungsten alloy, and the like.

It should be understood that although the above discussion was focused on percutaneous medical procedures within the vasculature of a patient, other examples or methods in accordance with the disclosure can be adapted and configured for use in other parts of the anatomy of a patient. For example, devices and methods in accordance with the disclosure can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the devices and methods described herein with respect to percutaneous deployment may be used in other types of surgical procedures as appropriate. For example, in some examples, the devices may be deployed in a non-percutaneous procedure. Devices and methods in accordance with the disclosure can also be adapted and configured for other uses within the anatomy.

It should be understood that this disclosure is, in many respects, only illustrative. Changes may be made in details, particularly in matters of shape, size, and arrangement of steps without exceeding the scope of the disclosure. The invention’s scope is, of course, defined in the language in which the appended claims are expressed.

What is claimed is:

1. An access sheath for a medical device, comprising:
an elongate shaft having a distal end region, a proximal end region and a wall;
a hub coupled to the proximal end region of the elongate shaft; and
a cutter releasably coupled to the hub, wherein the cutter is configured to translate along the elongate shaft from the proximal end region to the distal end region;
wherein translation of the cutter along the elongate shaft cuts the wall of the elongate shaft from the proximal end region to the distal end region.

2. The access sheath of claim 1, wherein the cutter includes a first grip member and a second grip member, wherein the first grip member includes a top surface and a bottom surface, and wherein the bottom surface faces a longitudinal axis of the elongate shaft.

3. The access sheath of claim 2, wherein the cutter further includes a blade assembly coupled to the bottom surface of the first grip member.

4. The access sheath of claim 3, wherein the blade assembly includes a blade having a cutting surface and guide member coupled to the blade.

5. The access sheath of claim 4, wherein the hub includes an inner aperture, and wherein the blade assembly is configured to be positioned within the aperture in a first position, and wherein the cutter is prevented from translating relative to the hub when the blade assembly is positioned within the aperture.

6. The access sheath of claim 5, wherein the first grip member is configured to shift from a first position to a second position, and wherein shifting the first grip member from the first position to the second position shifts the blade assembly out of the aperture and into an inner chamber of the hub.

7. The access sheath of claim 6, wherein shifting the blade assembly out of the aperture permits the cutter to translate relative to the hub, the elongate shaft or both the hub and the elongate shaft.

8. The access sheath of claim 7, wherein the guide member is configured to prevent the blade from being disengaged with the wall of the elongate shaft as the cutter translates along the elongate shaft.

9. The access sheath of claim 8, wherein the guide member includes a top surface configured to engage an inner surface of the elongate member as the cutter translates along the elongate shaft.

10. The access sheath of claim 2, wherein the cutter further includes a body having an aperture therein, and wherein the aperture is configured to permit the cutter to be removed from a medical device extending through at least a portion of the body.

11. The access sheath of claim 1, wherein the cutter includes a first groove and a second groove, and wherein the cutter is configured to be separated into a first cutter member and a second cutter member via tearing the cutter along the first groove and the second groove.

12. The access sheath of claim 1, wherein the hub is configured to separate into a first hub member and a second hub member.

13. The access sheath of claim 12, wherein the hub further includes a frangible region connecting a first hub member to a second hub member.

14. The access sheath of claim 13, wherein the frangible region may be configured to facilitate the separation of the hub into the first hub member and the second hub member.

15. The access sheath of claim 14, wherein the first hub member is coupled to the second hub member via a splitable membrane.

16. The access sheath of claim 1, further comprising a seal releasably coupled to the hub, wherein the seal is configured to be removed from a medical device extending through a portion of the seal.

17. An access sheath for a medical device, comprising:
an elongate shaft having a distal end region, a proximal end region and a wall;
a hub coupled to the proximal end region of the elongate shaft, wherein the hub includes a first hub member configured to separate from a second hub member;
a cutter releasably coupled to the hub, wherein the cutter is configured to translate along the elongate shaft from the proximal end region to the distal end region; and
a seal releasably coupled to the hub, wherein the seal is configured to be removed from a medical device extending through a portion of the seal;
wherein translation of the cutter along the elongate shaft cuts the wall of the elongate shaft from the proximal end region to the distal end region.

18. The access sheath of claim 17, wherein the cutter includes a first grip member and a second grip member, wherein the first grip member includes a top surface and a bottom surface, and wherein the bottom surface faces a longitudinal axis of the elongate shaft.

19. The access sheath of claim 18, wherein the cutter further includes a blade assembly coupled to the bottom surface of the first grip member.

20. A method for advancing a medical device into a body lumen, comprising:
positioning an access sheath adjacent a body lumen, the access sheath comprising:
an elongate shaft having a distal end region, a proximal end region and a wall;
a hub coupled to the proximal end region of the elongate shaft; and
a cutter releasably coupled to the hub, the cutter including a blade configured to cut the wall of the elongate shaft;
advancing the medical device through the access sheath and into the body lumen;
translating the cutter along the elongate shaft from the proximal end region to the distal end region, wherein translation of the cutter along the elongate shaft cuts the wall of the elongate shaft from the proximal end region to the distal end region; and
removing the access sheath from the medical device via passing at least a portion of the medical device through the cut portion of the elongate shaft.
